(19) United States
(12) Patent Application Publication
(10) Pub. No.: US 2024/0206945 A1
(43) Pub. Date: Jun. 27, 2024

(54) ELECTROSURGICAL GENERATOR

(71) Applicant: OLYMPUS WINTER & IBE GMBH, Hamburg (DE)

(72) Inventors: Daniel RAMIN, Nuthetal (DE); Thomas Fähsing, Berlin (DE)

(73) Assignee: OLYMPUS WINTER & IBE GMBH, Hamburg (DE)

(21) Appl. No.: 18/391,811

(22) Filed: Dec. 21, 2023

Related U.S. Application Data
(60) Provisional application No. 63/434,996, filed on Dec. 23, 2022.

(51) Int. Cl. A61B 18/12

(52) U.S. Cl. CPC A61B 18/1206; H03F 3/217

(57) ABSTRACT

An electrosurgical generator for outputting a HF voltage to an electrosurgical instrument includes a power supply unit, an inverter unit generating the HF voltage, a base module and a plurality of function modules for performing various functions of the electrosurgical generator, at least one thereof being configured for controlling a user interface. Each of the function modules features a galvanically separated local operating power supply includes a transformer being fed by the base module having an operating power distribution unit. A central AC generator is provided generating AC voltage supplied to the operating power distribution unit. The transformers are decentralized at each of the function modules. This combination of centralized AC generation and decentralized transformers increases efficiency, reduces space demand and facilitates effective shielding, thereby providing a more economical, compact and EMR improved electrosurgical generator.

The invention relates to an electrosurgical generator configured to output a high-frequency alternating voltage to an electrosurgical instrument. The electrosurgical generator comprises a power supply unit and an inverter unit generates a high-frequency alternating voltage to be supplied to at least one output socket configured for connection of the electrosurgical instrument.

In electrosurgery or high-frequency surgery, an electrosurgical instrument such as an electroscalpel is used to apply high-frequency alternating current to tissue in the human body. Usually high-frequency in the radiofrequency range of about 200 kHz to up to 4,000 kHz is used. This results in local heating of the tissue. Thereby, the tissue is cut or severed by heating, and the tissue is removed by thermal resection. A major advantage of this is that bleeding can be stopped at the same time as the cutting is made by closing the affected vessels, and electrosurgical instruments can be used for other applications, such as coagulation. However, a variant of electrosurgical generators exists that is capable of driving ultrasound instruments, employing high-frequency in the ultrasound frequency range, typically in the range of 20 kHz to 200 kHz (ultrasound surgical generators). Either way, electrosurgical generators are utilized to supply power to electrosurgical instruments and are designed to deliver a high-frequency alternating voltage to an electrosurgical instrument, so they are medical devices.

In order to provide electrosurgical generators for various fields of medicine, different types of electrosurgical generators are required having different components and featuring different functionality. There are various types of electrosurgical generators: apart from a type for general surgery various types exist, e.g., specialized for urology, gynecology, gastroenterology, pulmonology, ENT (ear, nose, throat), phlebology and visceral surgery just to name a few. Within each of these types there are also different variants of electrosurgical generators, some having lesser and others having a bigger range of functionality and power.

In order to provide these different functionalities, the electrosurgical generator is provided with various different functional entities (function modules). For the sake of patient safety they are supplied with electrical power that is galvanically isolated. Providing the different functional entities with galvancically isolated power is good practice and also usually required by applicable legislative or normative requirements.

However, providing such galvanically isolated power supplies for the different functional entities requires substantial effort in terms of component and costs. The number of electrical components scales with a higher number of function modules. This requires substantial efforts if a plurality of function modules are to be supplied. But not just effort and cost are increased, there is also an increase in electromagnet emissions. Due to each function module having its own AC generation, a lot of AC and therefore electromagnetic emissions are created. This leads to multiplication and nearly uncontrolled interference of different electromagnetic sources which is difficult to control and to keep reliably within acceptable limits, in particular concerning electromagnetic compatibility (EMC).

It is thus an object of the invention to provide an electrosurgical generator that reduces this drawback.

The solution according to the invention consists in an electrosurgical generator according to the features of the independent claim. Advantageous developments are the subject matter of the dependent claims.

In an electrosurgical generator configured to output a high-frequency alternating voltage to an electrosurgical instrument, comprising a power supply unit, an inverter unit generating a high-frequency alternating voltage to be output to the electrosurgical instrument, and a plurality of function modules configured for performing functions of the electrosurgical generator, at least one of the function modules being configured for conveying the high-frequency alternating voltage to the at least one output socket or for controlling a user interface of said electrosurgical generator, wherein each of the function modules is provided individually with a galvanically separated local operating power supply comprising a transformer, according to the invention a base module is provided comprising an operating power distribution unit feeding the local operating power supply of the plurality of function modules, and a central AC generator is provided, preferably at the base module, generating an AC voltage to be supplied to the operating power distribution unit, the transformers being provided decentralized at each of the function modules, wherein the operating power distribution unit is configured for a distribution of the AC voltage generated by the central AC generator to the function modules and their respective transformers.

In the following, some expressions that are used within the context of the invention are explained:

The inverter unit is a device to provide the actual high-frequency alternating voltage output for the surgical instrument to be connected to the output socket. The term inverter is rather broad and comprises actual inverter technology as well as converters and amplifiers.

In the context of the present application the term “high-frequency” relates it to frequencies in the radiofrequency range of 200 kHz to 4000 kHz as generated by the inverter of the electrosurgical generator. However, for electrosurgical generators being capable of driving ultrasound instruments, the term “high-frequency” also relates to the ultrasound frequency range, typically in the range of 20 kHz to 200 KHz (ultrasound surgical generators). The high frequency alternating voltage is typically high voltage. In the context of the present patent high voltage is considered as having an amplitudes in the high voltage range, in particular up to 10 kV, preferably up to 4000 Volt and further preferably more than 100 Volt.

The invention draws on the fact that by a combination of a single, centralized AC generator with an operating power distribution unit feeding the generated AC voltage to the local operating power supply of the function modules, the advantages of having galvanically separated function modules can be combined with the event advantage of lesser efforts by providing a centralized AC generator. Having a central AC generator supplying the various function modules with AC voltage has the benefit that only one single (centralized) AC generator is required as opposed to a plurality of distributed electromagnetic sources if AC generation were to be arranged individually at the different function modules as like in the prior art. Moreover, providing an electromagnetic shielding can be effected more efficiently for a single AC generator as opposed to providing shielding for plurality of AC generators being locally distributed on the individual function modules. Finally, and this is quite important for electrosurgical generators having many function modules, providing a central AC generator saves space as opposed to having a plurality of local AC generators and therefore allows for a more compact arrangement.

Another important advantage in particular for reliable operation is, that stabilization of the AC voltage generated can be effected more efficiently if it is only one single but more powerful AC source that needs to be stabilized as opposed to having various local smaller AC sources distributed on the individual function modules.

Finally, any effort required to enable the AC generator for production of a rather pure sinus AC voltage having low harmonic content needs to be spent only one time in case of a central AC generator as opposed to a plurality of AC generation units in case of local production at the function modules. This allows for more sophisticated generation of pure sinus waves, thereby allowing for an increase of efficient operation of subsequent transformers and for a decrease of electromagnetic emissions due to reduced harmonics.

Preferably, the central AC generator is configured to provide AC voltage having a frequency of at least 1 kHz, preferably 20 kHz to 100 kHz, preferably up to 70 kHz. Selecting a higher frequency, e.g., 20 kHz, features the advantage that subsequent transformers to be arranged on the plurality of function modules can be dimensioned smaller due to higher efficiency of the transformers if supplied with a high-frequency, thereby a smaller sized transformer suffices. Due to the plurality of transformers this provides for a considerable saving of space and costs.

Advantageously, the central AC voltage generator is provided with a central AC voltage stabilization unit. Having just one stabilizing unit at a rather powerful voltage generator allows for more efficient stabilization of the voltage as opposed to having a plurality of independent voltage stabilization units individually at each of the function modules. Further, due to the more powerful configuration of the single centralized AC generator voltage stabilization is facilitated. As a result, a more effective and powerful voltage stabilization can be achieved.

In a preferred variant thereof, a feedback circuit for actual voltage output by the transformer is provided as an input for the central voltage stabilization unit. This can be accomplished by a voltage measuring sensor, and its measurement signals are being fed back to an AC generator control unit. The feedback may be applied directly or indirectly, via a data bus (e.g., CAN). Thereby, any deviation in the output voltage of the transformer can be corrected by increasing or decreasing the amplitude of the output of the AC generator, thereby effecting a compensation. This provides for a stable voltage supply of the function modules even under demanding high load conditions, and therefore improves stability and reliability of operation of the function modules.

In an advantageous embodiment, an audio-type amplifier is provided for the central AC generator. Thereby, the invention takes advantage of audio amplifiers being well adapted to amplify sinus signals up to quite powerful levels without distortions. As a result, transformers being supplied by the audio amplifier can receive a sinus signal having a high quality with minimum distortions. Further, audio amplifiers are designed to operate in the kilohertz range and are enabled to deliver frequencies of 20 kHz or even more without substantial distortions. This allows for an efficient operation of the transformers. Moreover, audio amplifiers are readily available at rather low cost.

Preferably, the audio-type amplifier is a class-D amplifier. Such class-D amplifiers are operating very energy efficient and thus produce a desired sinus AC output with a minimum of energy consumption and heat generation. The latter is a rather significant advantage if a lot of function modules are placed in a compact casing of the electrosurgical generator. Preferably, the class-D amplifier is provided with a reconstruction filter. By having such a reconstruction filter, very high-frequency components like harmonics can be removed from the output signal of the class-D amplifier in order to achieve a continuous sinus signal having a high degree of purity, i.e. with reduced harmonics. However, utilizing a amplifier of the class-D type is not strictly necessary, other types of audio amplifiers or other linear amplifiers having sufficient output power can be employed too. The amplifier may be formed as an integrated circuit (IC), or may be based on a discrete design, or any combination thereof. The reconstruction filter is preferably arranged centrally, in particular at the amplifier. A central reconstruction filter has the benefit of requiring less components than decentral reconstruction filters on the function modules and further reduces unwanted RF emission.

Advantageously, as an output stage of the class-D amplifier a full-bridge is provided. Thereby, rather high output power can be provided. However, other types are possible, e.g., a half-bridge which complements the already high efficiency of the class-D amplifier (as opposed to a full-bridge output stage), and further allows to reduce the number of expensive high power semiconductor components, and to increase reliability due to reduced component count.

A control signal which is to be used as an input signal to be amplified by the audio-type amplifier may be an analog signal. This provides for a rather straightforward amplification, in particular if a linear amplifier is to be employed. However, preferably a control signal to be amplified by the audio-type amplifier is a digital signal, preferably a PCM signal. This allows for a pure digital path thereby avoiding any artifacts as they may be created by digital/analog conversion and vice versa. Further, the controlling signal can be furnished by a microprocessor, in particular a microprocessor of an operational control unit of the electrosurgical generator, as a digital signal. This has the advantage of providing the signal used for controlling directly, without any conversion. It is particularly beneficial if such digital signal is a PCM (pulse code modulation) signal, a 12S signal (Inter-IC Sound) or the like. A particular benefit of such a digital signal is that it can be readily output by a microprocessor and further it suits very well to the input of a digital amplifier, in particular a class-D amplifier. Combining such a digital, in particular, PCM signal as control signal for class-D amplifier provides for a streamlined, direct and extremely efficient embodiment of an AC generator.

Preferably, a local operating power supply of the function modules is provided with a supply voltage conditioning circuit, preferably a DC rectifier and/or a voltage stabilization circuit. Such a conditioning circuit is located behind the transformer in the respective function modules, so that voltage output by the transformer is to be treated by said conditioning circuit. Thereby, an efficient generation of DC voltage in addition to AC voltage can be achieved, and further various different voltages can be generated. Further preferably, the voltage can be stabilized. Optionally, a local voltage stabilization circuit can be provided on the function module, thereby meeting the increased stability requirements of any sensitive circuit on the function module.

In a preferred embodiment which may deserve independent protection, the base module further comprises a plurality of receptacles connected with the operating power distribution unit, said receptacles being configured to mechanically receive one of the function modules and making an electrical connection between the distribution unit and said function module in its inserted state. Thereby, the receptacles of the base module can be populated with various configurations of function modules as selected for specific type or variant of the electrosurgical generator. This facilitates building of an electrosurgical generator according to the needs of a specific type or variant. Various types of variants could thereby be manufactured all being provided with a proper AC supply voltage by the central AC generator on the base module. The function module itself just needs a transformer that produces a voltage as required by said function module, and any conditioning circuit if so required. Providing mechanical fixation and electrical connections at the receptacles has the advantage that by inserting a function module in one or another receptacle allows for an automatic establishment of electrical connections and sufficient mechanical fixation, thereby further facilitating manufacturing and increasing reliability. Further, one variant could be easily modified or reconfigured to another variant by changing any of the function modules to another function module.

The term “receptacle” is to be understood as an electrotechnical term meaning a receptacle for an electrical module. Often it may feature a structural slot or slit to accommodate the electrical module, but this is not necessary. It is configured to arrange for both, mechanical fixation and making electrical contact to the function module once it is placed in the receptacle.

Advantageously, the base module further comprises a high frequency power distributing unit being supplied by the high frequency alternating voltage output by the inverter, the receptacles being connected to operating power distribution unit, said receptacles being further configured to make a high power electrical connection between the high frequency power distribution unit and at least one of the function modules in its inserted state. Thereby, not only the operational power supply as described above but also high-frequency power as generated by the inverter of the electrosurgical generator can be distributed via the base module to the function modules. Obviously, this is of particular relevance for function modules which are socket modules being connected to an output socket, into which an electrosurgical instrument is to be plugged. By virtue of this, operational power as well as the high frequency power can be delivered via the base module at once upon inserting the corresponding socket module into the receptacle.

A socket module is a module to be attached to the base module and being configured to supply the output socket with the high-frequency alternating voltage as generated by the inverter unit.

In a preferred embodiment, the base module further comprises a data communication distribution unit, the receptacles being further configured to make a data connection to at least one of the function module in its inserted state. By virtue of this, also a data connection to and from the function module can be established by inserting said function module into the respective receptacles.

The invention is explained in more detail below by way of examples in conjunction with the accompanying drawing, showing advantageous embodiments. In the drawing:

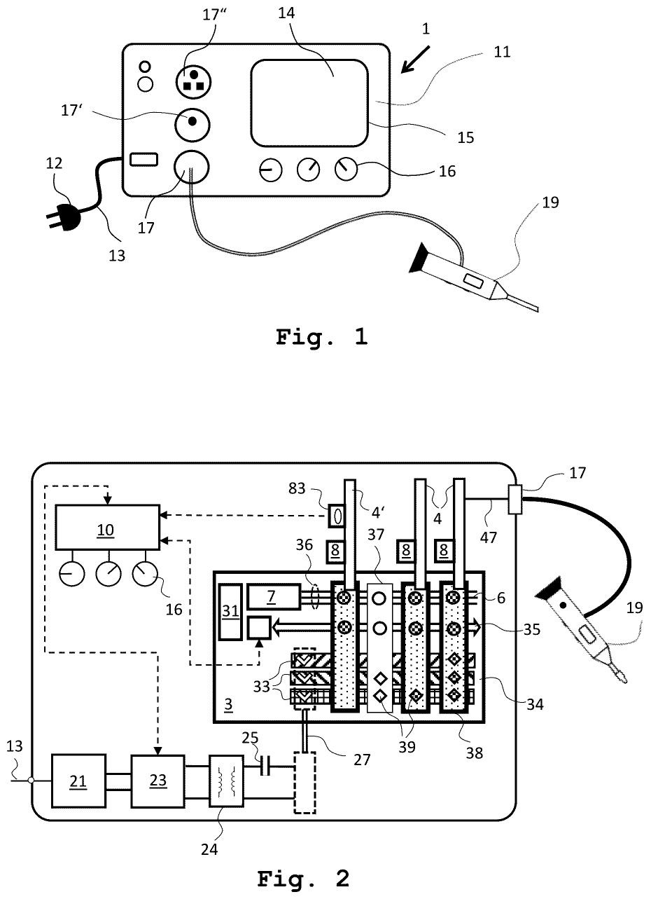

FIG. 1 shows a frontal view of an electrosurgical generator with an attached electrosurgical instrument;

FIG. 2 shows a schematic functional diagram of the electrosurgical generator including a base module;

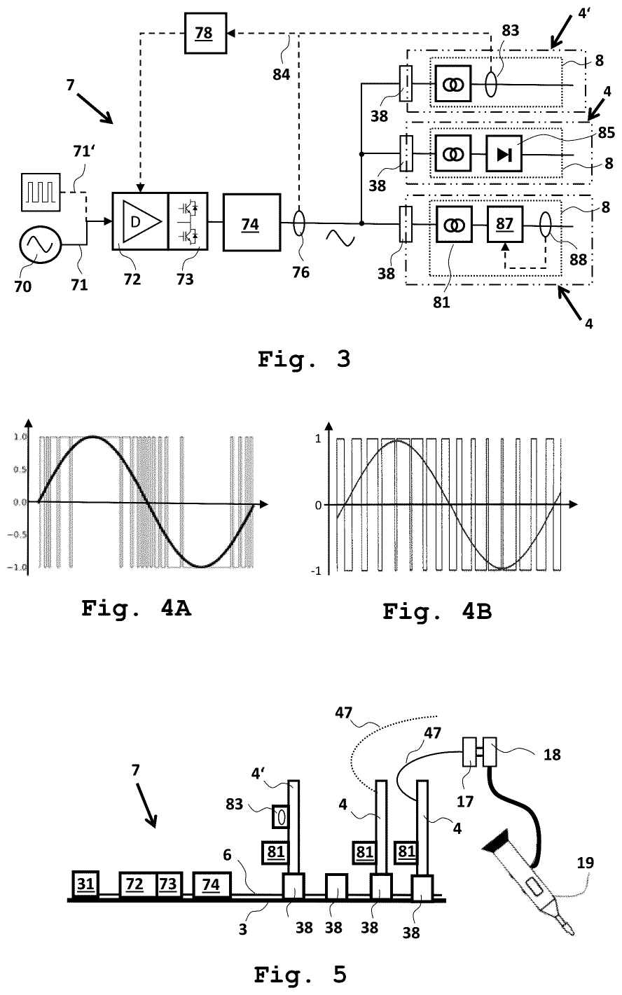

FIG. 3 shows a schematic operating power supply for function modules of the electrosurgical generator;

FIG. 4A, 4B show PWM and PCM coded sinusoidal signal; and

FIG. 5 shows a lateral view of the base module with inserted function modules.

An electrosurgical generator according to an exemplary embodiment of the invention is illustrated in FIG. 1. The electrosurgical generator, identified as a whole by reference numeral 1, comprises a housing 11 having at least one output socket 17 (and optionally additional output socket 17′ and 17″) for connection of an electrosurgical instrument 19. A power supply cable 13 is provided with a plug 12 for connection to an electrical power source (not shown) which may be an electricity grid, like AC mains in a building, or an off-grid source of electric energy, like a 12 Volt or 24 Volt battery in a vehicle or mobile hospital. Further, a user interface 14 is provided comprising a display 15 which may be a touchscreen, or knobs 16 may be present for inputs by a user.

Said electrosurgical instrument 19 comprises a cable with a plug 18 which is to be plugged-in into the output socket 17 in order to supply high-frequency alternating voltage for the electrosurgical instrument 19 which in the depicted exemplary embodiment is an electroscalpel.

FIG. 2 shows a schematic functional diagram of the electrosurgical generator. Electric power is delivered by the power supply cable 13 to a power supply unit 21 of the electrosurgical generator. The power supply unit provides electrical power, usually DC power, to the various components, units and modules of the electrosurgical generator 1. In particular, it provides electrical power to an inverter unit 23 configured for generating high-frequency alternating voltage that is fed to a step-up transformer 24 in order to deliver high voltage to the electrosurgical instrument 19, the high-voltage usually being in a range of a few kilovolts, but may have amplitudes in a range of several 10 Volt up to 4000 Volt.

The kind of inverter unit 23 is selectable by the person skilled in the art since there are several concepts known in the art, for example a forward converter or a multilevel inverter. The key point is that the inverter unit 23 generates the high-frequency alternating voltage in a voltage range high enough for proper operation of the electrosurgical instrument 19, which may as already indicated be as high as 4000 Volt. Operation of the inverter unit 24 is governed by a control unit 10 which in turn is connected with the user interface 14 such that the user can issue directions and commands for operation of the electrosurgical generator 1. The control unit 10 generates corresponding control signals and governs the relevant components, units and modules of the electrosurgical generator 1 according to these instructions and commands. This is generally known in the art and therefore need not to be described in more detail.

The high-frequency high voltage output generated by the inverter unit 23 and the step-up transformer 24 is routed via a blocking capacitor 25 to an output connector cable 27 and via a base module 3 and function modules 4 being plugged into receptacles 38 of the base module and ultimately via an interconnection cable 47 to the output socket 17.

Configuration of the base module 3 and the function modules 4 will be explained next taking reference to FIGS. 2 and 3. The base module 3 is configured with a plurality of receptacles 38 as already mentioned for insertion of various function modules 4. The function modules 4 for may serve different purposes, in particular function modules 4 being configured as a socket modules which are being designed to supply the high-frequency high voltage as a generated by the inverter 23 with the step-up transformer 24 to the output socket 17 in order to supply a plugged in electrosurgical instrument 19, or another kind of function module may be a function module 4′ controlling the user interface 14 and co-operating with the control unit 10.

For proper operation, the function modules 4 (and 4′) require an operating power supply. For this purpose, a local operating power supply 8 is provided individually on the respective function modules 4. Power is delivered by means of an operating power distribution unit 6 of the base module 3. Said distribution unit 6 comprises a set of conductors being arranged like a bus bar 36 for supplying the operating power to the function modules 4. The base module 3 further is provided with a data communication distribution unit 35 that comprises a data bus bar running parallel to the bus bar 36 of distribution unit 6. It can be configured as being a CAN (controller area network) bus in order to distribute signals and data communication between various function modules 4, the base module 3, the control unit 10, and other modules and units of the electrosurgical generator 1.

Further, on the base module 3 a high-frequency distribution unit 34 is provided to which the high-frequency alternating voltage is supplied from the inverter 23 via the connector cable 27.

The high-frequency distribution unit 34 comprises a plurality, in the depicted embodiment three, stripe conductors 33 configured for high-frequency and high-voltage. They comprise a first conductor for an active electrode, a second conductor being configured as neutral electrode, and a third conductor being configured as a second active electrode in the depicted exemplary embodiment. The stripe conductors 33 are dimensioned wider than the conductors of the operating power distribution unit 6 and are separated from each other by a spacing sufficient for high-voltage isolation as it is required for medical devices. Thereby, the high-frequency high voltage as generated by the inverter unit 23 with the step-up transformer 24 and supplied by the connector cable 27 is directly supplied to the stripe conductors 33 of the high-frequency distribution unit 34.

Further, on the base module 3 a plurality of receptacles 38 are provided. Each of the receptacles 38 comprises a guide 37 and one or more connection ports 39 forming contact to the high-frequency distribution unit 34, to the data distribution unit 35 and to the operating power distribution unit 6. The guide 37 of each of the receptacles 38 is configured to mechanically receive one of the function modules 4, wherein said function module 4 in its inserted state is electrically connected to the high-frequency distribution unit 34, to the data distribution unit 35 and to the operating power distribution unit 6 via said connection ports 39. This connection is performed automatically upon inserting the respective function module 4 in one of the receptacles 38.

Further, a microprocessor 31 is provided on the base module. It is configured for controlling operation of the base module 3 and further interacts with the control unit 10 of the electrosurgical generator 1 or may even perform processing tasks thereof.

For supplying operating power to the function module via the operating power distribution unit 6, a centralized AC generator 7 is provided. In the depicted embodiment it is provided on the base module 3. The AC generator 7 is configured to provide a sinusoidal AC voltage that can be supplied via the operating power distribution unit 6 to the function modules 4 placed in the receptacles 38 and being connected by the connection ports 39. On the function modules 4 a local operating power supply 8 is provided comprising a transformer 81 which is configured to transform the voltage supplied from the centralized AC generator 7 as required by the respective function module 4 and to provide a provide a galvanic isolation of the function module 4 with respect to the base module 3 and with respect to other function modules 4.

The AC generator 7 comprises a class-D amplifier 72 having an output stage 73 configured as a half bridge (or as a full bridge, in particular for higher output power). As an input signal for the class-D amplifier 72 a sinus signal is supplied. That sinus signal may be provided either by a dedicated sinus source 70 generating an analogue sinus signal 71 that is supplied to an input of the class-D amplifier 72, or as a preferred alternative a digital sinus signal 71′ which is supplied directly to the input of the class-D amplifier 72.

The digital sinus signal 71′ could be one of a variety of digital control signals, in particular it may be an I2C (Inter-IC Sound), I2c (Inter-Integrated Circuit), SPI (Serial Periphery Interface) or a pulse width modulated (PWM) signal. An example for such a PWM signal of the sinus is shown in FIG. 4a. In a preferred alternative, the digital sinus signal 71′ is a pulse code modulated (PCM) signal. An example for such a PCM signal is shown in FIG. 4b. As it can be readily appreciated, despite both signals being one bit signals, the PCM signal provides for a more accurate representation of the sinus wave. A further advantage of such a PCM signal is, that it can be readily generated and outputted by a microprocessor, so that a full digital signal chain can be accomplished, with the microprocessor 31 outputting the digital sinus signal 71′ to be supplied to the input of the class-D amplifier 72. The frequency of the sinus signal 71, 71′ is preferably about 20 KHz. Such a frequency is perfectly manageable by a class-D amplifier that is being capable of amplifying frequencies of the audio range, and further a frequency that high is beneficial in terms of efficiency and compactness of the transformers 81 located on the individual function modules 4.

The corresponding amplified signal as output by the class-D amplifier 72 and its output stage 73 is supplied to a reconstruction filter 74. It acts like a low-pass filter and is configured to remove higher frequency components, in particular harmonics, from the output of the class-D amplifier 72 in order to achieve a sinusoidal AC voltage with rather low harmonic content. This sinusoidal AC voltage is supplied to the distribution unit 6 for powering the local supply 8 of the individual function modules 4.

The sinusoidal AC voltage is conveyed via the distribution unit 6, the receptacles 38 and the function modules 4 inserted into the receptacles 38 to a primary side of the transformer 81 of the respective local operating power supply 8 of the individual function modules 4. The transformer 81 utilizes this high-frequency sinusoidal AC voltage to generate a galvanically isolated output AC voltage at the secondary side.

Thereby the required galvanic isolation can be achieved. Further, the transformers 81 a can be dimensioned according to the needs of the respective function module 4, thereby tailoring the local operating power supply 8 to the needs of the respective function module 4 in terms of output power and voltage.

For voltage stabilization, a voltage sensor 76 may be provided at the distribution unit 6. Its measurement signal is supplied to a voltage stabilization unit 78 that is provided for the central AC generator 7. The voltage stabilization unit 78 outputs a correction signal to the class-D amplifier 72 and is configured to adjust an amplification factor of the class-aD amplifier 72. Said correction signal may be applied directly or via a data bus (e.g., CAN) to the class-D amplifier.

Thereby, any sag/surge of the voltage of the distribution unit 6 can be compensated right at the source.

Further, the local operating power supply 8 may be provided with additional supply voltage circuitry. For example, the local operating power supply 8 of at least one function module 4 may have a rectifier 85 connected to the secondary side of the transformer 81 in order to provide a DC supply for components of said function module 4. Alternatively or additionally, the function module 4 may be provided with a local voltage stabilizing circuit 87 which monitors the local output voltage by means of a voltage sensor 88 and compensates for any deviation from a set reference voltage value.

Moreover, a feedback 84 having a voltage sensor 83 at the secondary side of the local transformer 81 may be provided, said feedback 84 supplying a measured signal of the local voltage on the respective function module 4 to the feedback circuit 78. Thereby central voltage stabilization can be achieved taking into account the voltage deviation as it is encountered on board of the local operating power supply at the respective function module 4. This allows for a flexible and efficient supply of operating power to the function modules tailored to their respective needs.

In FIG. 5 a lateral view of the base module 3 is shown. In the left portion of the figure the central AC generator 7 is shown. It comprises the class-D amplifier 72 with a digital input signal supplied by the microprocessor 31 and with its output stage 73 that is connected to the reconstruction filter 74. The sinusoidal AC voltage thereby formed is supplied via the operating power distribution unit 6 to the receptacles 38, and to the function modules 4 being inserted into the receptacle 38. Thereby, the local operating power supply units 8 of the function modules 4 with their respective transformer 81 are being supplied at their primary side with the AC voltage. On a secondary side of the transformer 81 galvanically isolated AC voltage is output for the power supply of the function modules 4. Further, as shown on the leftmost function module 4′, a voltage sensor 83 is provided which measures the output voltage on the secondary side of the transformer 81. Its measurement signal is supplied via the feedback 84 to the central stabilization unit 78 (ref. FIG. 3) in order to provide a stabilized supply voltage on the function modules 4 even under high load conditions. For feeding back of the measurement signal by the data communication distribution unit 35 may be employed.

1. An electrosurgical generator configured to output a high-frequency alternating voltage to an electrosurgical, instrument, comprising a power supply unit, an inverter unit generating a high-frequency alternating voltage to be output to the electrosurgical instrument, and a plurality of function modules configured for performing functions of the electrosurgical generator, at least one of the function modules being configured for conveying the high-frequency alternating voltage to the at least one output socket or for controlling a user interface of said electrosurgical generator,
wherein each of the function modules is provided individually with a galvanically separated local operating power supply comprising a transformer
wherein
a base module is provided comprising an operating power distribution unit feeding the local operating power supply of the plurality of function modules, and
a central AC generator is provided, generating an AC voltage to be supplied to the operating power distribution unit
the transformers being provided decentralized at each of the function modules, wherein the operating power distribution unit is configured for a distribution of the AC voltage generated by the central AC generator to the function modules and their respective transformer.

2. The electrosurgical generator of claim 1, wherein the central AC generator is configured to provide AC voltage having a frequency of at least 1 KHz.

3. The electrosurgical generator of claim 1, wherein a central AC voltage stabilization unit is provided for the central AC generator.

4. The electrosurgical generator of claim 3, wherein a feedback circuit for actual voltage output by the transformer is provided as an input for the central voltage stabilization unit.

5. The electrosurgical generator according to claim 1, wherein an audio-type amplifier is provided for the central AC generator.

6. The electrosurgical generator according to claim 5, wherein the audio-type amplifier is a class-D amplifier.

7. The electrosurgical generator according to claim 6, wherein the class-D amplifier operates a full-bridge as an output stage for the generated AC voltage.

8. The electrosurgical generator according to claim 5, wherein a control signal to be amplified by the audio-type amplifier is an analogue signal.

9. The electrosurgical generator according to claim 5, wherein a control signal to be amplified by the audio-type amplifier is a digital signal.

10. The electrosurgical generator of claim 9, wherein the control signal is provided digitally by a microcontroller.

11. The electrosurgical generator according to claim 1, wherein the local operating power supply of the function modules is provided with a supply voltage conditioning, preferably a DC and/or a voltage stabilization circuit.

12. The electrosurgical generator according to claim 1, wherein the base module further comprising a plurality of receptacles connected with the distribution unit, the receptacles being configured to mechanically receive one of the function modules and making an electrical connection between the distribution unit and the function module in its inserted state.

13. The electrosurgical generator according to claim 12, wherein the base module further comprises a high frequency power distributing unit being supplied by the high frequency alternating voltage output by the inverter, the receptacles being connected to the high frequency power distribution unit, the receptacles being further configured to make a high power electrical connection between the high frequency power distribution unit and at least one of the function modules in its inserted state.

14. The electrosurgical generator of claim 12, wherein the base module further comprises a data communication distribution unit, the receptacles being further configured to make a data connection to at least one of the function modules in its inserted state.

15. The electrosurgical generator configured to output the high-frequency alternating voltage to the electrosurgical instrument, comprising the power supply unit, the inverter unit generating the high-frequency alternating voltage to be output to the electrosurgical instrument, and the plurality of function modules configured for performing functions of the electrosurgical generator, at least one of the function modules being configured for conveying the high-frequency alternating voltage to the at least one output socket or for controlling the user interface of said electrosurgical generator,
wherein each of the function modules is provided individually with the galvanically separated local operating power supply comprising the transformer
wherein
the base module is provided comprising the operating power distribution unit feeding the local operating power supply of the plurality of function modules, and
the base module, generating the AC voltage to be supplied to the operating power distribution unit
the transformers being provided decentralized at each of the function modules, wherein the operating power distribution unit is configured for the distribution of the AC voltage generated by the central AC generator to the function modules and their respective transformer.

16. The electrosurgical generator of claim 9, wherein the control signal is provided digitally by the microcontroller of an operational control unit of the electrosurgical generator.

17. The electrosurgical generator according to claim 1, wherein the local operating power supply of the function modules is provided with a DC rectifier and/or a voltage stabilization circuit.
